(19) United States
(12) Patent Application Publication
(10) Pub. No.: US 2022/0142905 A1
(43) Pub. Date: May 12, 2022

(54) COSMETIC PRODUCT CONTAINING A MIXTURE OF SILICONES

(71) Applicant: Henkel AG & Co. KGaA, Duesseldorf (DE)

(72) Inventors: Rene KROHN, Norderstedt (DE); Thomas HIPPE, Appen (DE); Stefan HOEPFNER, Hamburg (DE); Jessica BRENDER, Hamburg (DE)

(73) Assignee: Henkel AG & Co. KGaA, Duesseldorf (DE)

(21) Appl. No.: 17/438,881

(22) PCT Filed: Feb. 27, 2020
(86) PCT No.: PCT/EP2020/055081

§ 371 Date: Sep. 13, 2021

(30) Foreign Application Priority Data
Mar. 12, 2019 (DE) 10 2019 203 297.4

(51) Int. Cl. A61K 8/898; A61K 8/892; A61K 8/34; A61Q 5/12

(52) U.S. Cl. CPC A61K 8/898; A61K 8/892; A61K 2800/33; A61Q 5/12; A61K 8/342

(57) ABSTRACT

The present disclosure present disclosure relates to a cosmetic product comprising a hydroxy-terminated polyorganosiloxane, a reaction product of a hydroxy-terminated polyorganosiloxane with an acid and/or an alcohol and/or a wax, and an aqueous or aqueous-alcoholic carrier.

### CROSS-REFERENCE TO RELATED APPLICATION

This application is a U.S. National-Stage entry under 35 U.S.C. § 371 based on International Application No. PCT/EP2020/055081, filed Feb. 27, 2020, which was published under PCT Article 21(2) and which claims priority to German Application No. 102019203297.4, filed Mar. 12, 2019, which are all hereby incorporated in their entirety by reference.

### TECHNICAL FIELD

The present disclosure present disclosure relates to cosmetic products comprising a mixture of at least two silicones.

There is a need to further improve hair care products and give them more beneficial properties. In particular, they should provide better care for the hair surface.

### BACKGROUND

Environmental influences as well as oxidative, reductive and/or surfactant hair treatments often lead to hair damage. Such damage manifests itself, for example, in impaired combability of the dry and wet hair. Moreover, the gloss and moisture balance are adversely affected by the attacked outer structure of the hair. Conventional cosmetic care products therefore contain conditioning polymers, such as cationic polymers or silicone oils.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It is therefore an object of the present disclosure present disclosure to reduce the side effects/damage of environmental influences and of oxidative, reductive and/or surfactant hair treatments and to condition the hair.

Surprisingly, it has now been found that the above-mentioned challenges are solved to an excellent degree by a cosmetic agent comprising two selected silicones.

Such a cosmetic agent provides intensive conditioning of keratinous fibers, especially human hair.

A first object of the present disclosure present disclosure is therefore a cosmetic agent for the treatment of keratinous fibers, comprising (relative to the weight of the entire agent)

a) 0.1 to 10% by weight of a hydroxy-terminated polyorganosiloxane,
b) 0.1 to 10% by weight of a reaction product of a hydroxy-terminated polyorganosiloxane with an acid and/or a fatty alcohol and/or a wax, and
c) an aqueous or aqueous-alcoholic carrier.

In principle, keratin fibers are understood to include all animal hair, for example wool, horsehair, angora hair, furs, feathers and products or textiles made from them. We are addressing here, however, human hair in particular.

In a preferred form of the present disclosure present disclosure, the cosmetic agents are used for the care/conditioning of keratinous fibers, in particular human hair. Therefore, conditioning agents such as hair conditioners or hair treatments are preferred cosmetic agents.

An oxidative hair treatment is defined as the action on keratinous fibers of an oxidative cosmetic agent comprising, in a cosmetic carrier, at least one oxidizing agent.

A reductive hair treatment is defined as the action on keratinous fibers of a reductive cosmetic agent comprising, in a cosmetic carrier, at least one reducing agent.

Surfactant treatment is defined as the action on keratinous fibers of a surfactant-comprising cosmetic agent comprising, in a cosmetic carrier, at least one surfactant, preferably an anionic surfactant.

The cosmetic agent comprising a combination of two selected silicones is preferably used immediately after oxidative, reductive and/or surfactant treatment. For the purposes of the present disclosure, after the oxidative, reductive or surfactant treatment is understood to mean an application which either directly follows the oxidative, reductive or surfactant treatment of keratinous fibers, the cosmetic agent being applied to the keratinous fibers, which are preferably still wet and towel-dry, after the oxidative, reductive or surfactant agent has been rinsed off, or being applied to the wet keratinous fibers only after several hours. The cosmetic agent can be rinsed out again after an exposure time of a few seconds up to 45 minutes, or it can remain completely on the keratinous fibers. It is preferred that the cosmetic agent is rinsed with water after the exposure time.

The cosmetic product comprises as ingredient a) a hydroxy-terminated polyorganosiloxane.

Known hydroxy-terminated polyorganosiloxanes are those with the INCI designation dimethiconol.

It may be preferred that the hydroxy-terminated polyorganosiloxane is selected from at least one compound of formulae (I) and/or (II)

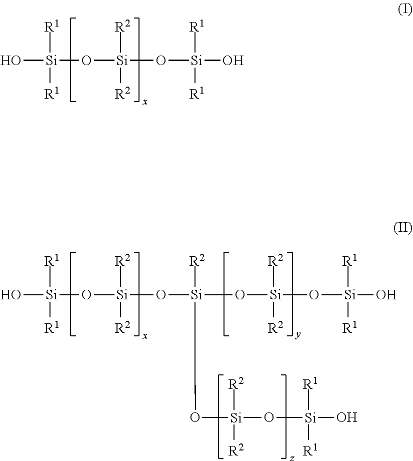

whereby in the formulas (I) and (II)
R1 represents hydrogen, a methyl radical, a C2 to C30 linear, saturated or unsaturated hydrocarbon radical, a phenyl radical and/or an aryl radical,
R2 represents hydrogen, a methyl radical, a C2 to C30 linear, saturated or unsaturated hydrocarbon radical, a phenyl radical and/or an aryl radical, and
x, y and z each independently represent an integer from 0 to 50,000

Particularly suitable hydroxy-terminated polyorganosiloxanes are polyorganosiloxanes of formula (I)

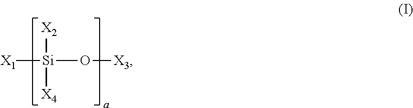

wherein
X1 and X2 are independently OH, OR1, R2, O-PDMS or O-fSiloxane,
X3 is hydrogen or a monovalent hydrocarbon radical having 1 to 8 carbon atoms per radical, PDMS or fsiloxane,
X4 is a remainder of the formula

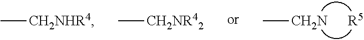

and
a is a number from 1 to 100, preferably a number from 1 to 5,
where
R1 is an alkyl radical having 1 to 8 carbon atoms,
R2 denotes a monovalent, saturated or unsaturated hydrocarbon radical which is optionally substituted by the elements N, P, S, O, Si and halogen and has 1 to 200 carbon atoms per radical,

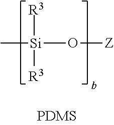

means,

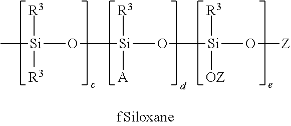

means,
R3 independently of one another is in each case a monovalent saturated or unsaturated hydrocarbon radical having 1 to 200 carbon atoms per radical and optionally substituted by the elements N, P, S, O, Si and halogen,
A is a radical of the formula R6-[NR7-R8-]fNR72,
where
R6 represents a divalent linear or branched hydrocarbon radical comprising 3 to 18 carbon atoms,
R7 represents a hydrogen atom, an alkyl group having 1 to 8 carbon atoms or an acyl group,
R8 is a divalent hydrocarbon radical having 1 to 6 carbon atoms,
b is a number from 1 to 2000, preferably from 1 to 1000,
c is 0 or a number from 1 to 2000, preferably from 50 to 1000,
d is a number from 1 to 1000, preferably from 1 to 10,
e is 0 or a number from 1 to 5,
f is 0, 1, 2, 3 or 4,
Z is hydrogen, an alkyl radical having 1 to 8 carbon atoms or

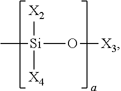

R4 represents a monovalent hydrocarbon radical optionally comprising N and/or O atoms and having 1 to 18 carbon atoms, and
R5 represents a divalent hydrocarbon radical optionally comprising N and/or O atoms and having 3 to 12 carbon atoms,
with the proviso that the polyorganosiloxane of formula (I) has at least one terminal OH group.

Examples of an alkyl radical R1 are methyl, ethyl, n-propyl, iso-propyl, 1-n-butyl, 2-n-butyl, iso-butyl, tert.-butyl, n-pentyl, iso-pentyl, neo-pentyl, tert.-butyl, n-hexyl, n-heptyl, n-octyl, iso-octyl or 2,2,4-trimethylpentyl, with methyl, ethyl and butyl being preferred.

Examples of hydrocarbon radicals R2 and R3 are alkyl radicals, such as methyl, ethyl, n-propyl-, iso-Propyl-, 1-n-butyl-, 2-n-butyl-, iso-Butyl-, tert.-butyl-, n-pentyl-, iso-pentyl-, neo-pentyl-, tert.-pentyl, n-hexyl, n-heptyl, n-octyl, iso-octyl, 2,2,4-trimethylpentyl-, n-nonyl-, n-decyl-, n-dodecyl-, n-octadecyl-, cyclopentyl-, cyclohexyl-, cycloheptyl-, methylcyclohexyl-, vinyl-, 5-hexenyl-, cyclohexenyl-, 1-propenyl-, allyl-, 3-butenyl-, 4-pentenyl-, phenyl, naphthyl, anthryl, phenanthryl, o-tolyl, m-tolyl, p-tolyl, xylyl, ethylphenyl, benzyl, alpha-phenylethyl and beta-phenylethyl radicals. Preferred R2 radicals are the methyl, ethyl, octyl and phenyl radicals, and particularly preferred are the methyl and ethyl radicals.

Examples of halogenated R2 and R3 radicals include the 3,3,3-trifluoro-n-propyl, 2,2,2,2′,2′,2′-hexafluoroisopropyl, heptafluoroisopropyl, o-chlorophenyl, m-chlorophenyl and p-chlorophenyl radicals.

Examples of R4 include the alkyl, cycloalkyl, aryl, alkaryl and aralkyl radicals listed for hydrocarbon radicals R2 and R3.

Preferred examples of R5 are radicals of the formulae —CH2-CH2-O—CH2-CH2-, —CH2-CH2-NH—CH2-CH2- or —CH2-CH2-NH—CH2-, the radical —CH2-CH2-O—CH2-CH2— being particularly preferred.

Examples of R6 are alkylene radicals with 3 to 10 carbon atoms such as propylene, butylene, pentylene, hexylene, heptylene, octylene, nonylene and decylene.

R7 may be a hydrogen, a methyl, ethyl, n-propyl, iso-propyl, 1-n-butyl, 2-n-butyl, iso-butyl, tert.-butyl, n-pentyl, iso-pentyl, neo-pentyl, tert.-butyl, n-hexyl, n-heptyl, n-octyl, iso-octyl, 2,2,4-trimethylpentyl or acetyl, with a hydrogen atom being preferred.

Preferred examples of R8 include alkylene radicals such as methylene, ethylene, propylene, butylene, pentylene, or hexylene.

Z is preferably hydrogen or methyl, ethyl, n-propyl, iso-propyl, 1-n-butyl, 2-n-butyl, iso-butyl, tert.-butyl, n-pentyl, iso-pentyl, neo-pentyl, tert.-butyl, n-hexyl, n-heptyl, n-octyl, iso-octyl or 2,2,4-trimethylpentyl, with hydrogen, methyl, ethyl and butyl being particularly preferred.

Preferred X4 radicals according to the above definitions for R4 and R5 are aminomethyl, methylaminomethyl, dimethylaminomethyl, diethylaminomethyl, dibutylaminomethyl, cyclohexylaminomethyl, morpholinomethyl, piperidinomethyl, piperazinomethyl, ((diethoxymethylsilyl)methyl)cyclohexylaminomethyl, ((triethoxysilyl)methyl)cyclohexylaminomethyl, anilinomethyl, 3-dimethylaminopropylaminomethyl, to (3-dimethylaminopropyl)aminomethyl and mixtures thereof. In this context, it is highly preferred if the cosmetic agent comprises polyorganosiloxanes of the formula (I) which contain the morpholinomethyl radical as radical X4.

According to the definitions for R6, R7 and R8 are preferred examples of radical A:

### —(CH2)3NH2

### —(CH2)3-NH—(CH2)2-NH2

### —CH2CH(CH3)CH2-NH—(CH2)2-NH2

### —(CH2)3-NH(Cyclohexyl)

### —(CH2)3-NHCH3

### —(CH2)3-N(CH3)2

### —(CH2)3-NHCH2CH3

### —(CH2)3-N(CH2CH3)2

### —(CH2)4-NH2

### —CH2CH(CH3)CH2-NH2

### —(CH2)3-NH—(CH2)2-NHCH3

### —(CH2)3-NH—(CH2)2-N(CH3)2

### —(CH2)3-NH—(CH2)2-NHCH2CH3

### —(CH2)3-NH—(CH2)2-N(CH2CH3)2

### —(CH2)3[-NH—CH2CH2]2-NH2

### —(CH2)3-NH(acetyl)

### —(CH2)3-NH—(CH2)2-NH(acetyl) and

### —(CH2)3-N(Acetyl)-(CH2)2-NH(Acetyl).

For the preparation of the polyorganosiloxanes of the formula (I), preferably commercially available polydimethylsiloxanes having terminal silanol groups and/or polydimethylsiloxanes having terminal alkoxy and silanol groups and/or amine-functionalized siloxanes comprising silanol groups or alkoxy and silanol groups are reacted with a dialkoxy- and/or trialkoxysilane which has a radical formula

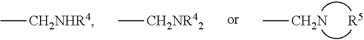

Accordingly, in formula (I), “fSiloxane” represents a radical derived from an amine-functionalized siloxane.

Trialkoxysilanes or a mixture of dialkoxy- and trialkoxysilanes, are particularly preferred, with the sole use of trialkoxysilanes being especially preferred. When trialkoxysilanes or a mixture of dialkoxy- and trialkoxysilanes are used, at least partially crosslinked polyorganopolysiloxanes are obtained, regardless of the structure of the siloxanes used and the position of the alkoxy and/or silanol groups in the siloxanes. In a very particularly preferred embodiment, the cosmetic product comprises crosslinked polyorganosiloxanes. In a highly preferred embodiment, the cosmetic product comprises crosslinked polyorganosiloxanes derived from the reaction of siloxanes and trialkoxysilanes.

Preferred examples of the dialkoxy or trialkoxysilanes used include:

### Diethylaminomethylmethyldimethoxysilane,

### Dibutylaminomethyltriethoxysilane,

### Dibutylaminomethyltributoxysilane,

### Cyclohexylaminomethyltrimethoxysilane,

### Cyclohexylaminomethyltriethoxysilane,

### Cyclohexylaminomethyl-methyldiethoxysilane,

### Anilinomethyltriethoxysilane,

### Anilinomethylmethyldiethoxysilane,

### Morpholinomethyltriethoxysilane,

### Morpholinomethyltrimethoxysilane,

### Morpholinomethyltriisopropoxysilane,

### 3-Dimethylaminopropyl-aminomethyltrimethoxysilane,

### Morpholinomethyltributoxysilane,

Morpholinomethyltrialkoxysilane, the alkoxy radical being a C1-C4-alkoxy radical, in particular a mixture of methoxy and ethoxy radicals,
piperazinomethyltriethoxysilane,
piperidinomethyltriethoxysilane and
partial hydrolysates thereof.

A particularly preferred silane is morpholinomethyltriethoxysilane.

A particularly preferred amine-functionalized siloxane is a copolymer of 3-(2-aminoethylamino)propylmethylsiloxy and dimethylsiloxy units having silanol groups or alkoxy and silanol groups.

A cosmetic product is particularly preferred in which at least one compound known under the INCI designation amodimethicone/morpholinomethyl silsesquioxane copolymer is used as the hydroxy-terminated polyorganosiloxane. This polyorganosiloxane is commercially available under the name Belsil® ADM 8301 E (ex Wacker). The raw material is a microemulsion and has the following components: Amodimethicone/morpholinomethyl silsesquioxane copolymer, trideceth-5, glycerin, phenoxyethanol and water.

The use of the hydroxy-terminated polyorganosiloxanes described above is preferably as aqueous suspensions or aqueous emulsions of hydroxy-terminated polyorganosiloxanes. The dispersions may contain one or more surfactants as dispersants. The surfactants are preferably non-ionic. Alternatively, inorganic solids such as silicas and/or bentonites may be used as dispersants. The average particle size of the polyorganosiloxanes measured by light scattering in the dispersions is preferably in the range 0.001 to 100 μm, more preferably 0.002 to 10 μm. The pH values can vary from 1 to 14. Preferably the pH is from 3 to 9, more preferably from 5 to 8.

The cosmetic product comprises the hydroxy-terminated polyorganosiloxane in an amount of from 0.1 to 10% by weight, preferably from 0.1 to 8% by weight, more preferably from 0.125 to 6% by weight, still more preferably from 0.15 to 4% by weight and very particularly preferably from 0.2 to 2% by weight, in each case based on the weight of the cosmetic product.

A second essential ingredient b) in the cosmetic product is a reaction product of a hydroxy-terminated polyorganosiloxane with an acid and/or an alcohol and/or a wax.

The acid, alcohol or wax react with the terminal hydroxyl group(s) of polyorganosiloxane to form, for example, ester or ether.

The acid is preferably including comprising selected from the group of fatty acids, amino acids, α-hydroxy acids and mixtures thereof.

Accordingly, it is preferred that the agent comprises, as a reaction product of a hydroxy-terminated polyorganosiloxane with an acid, a compound including comprising selected from the group of reaction products of a hydroxy-terminated polyorganosiloxane with a fatty acid, reaction products of a hydroxy-terminated polyorganosiloxane with an amino acid, reaction products of a hydroxy-terminated polyorganosiloxane with an α-hydroxy acid, and mixtures thereof.

In the context of the present disclosure present disclosure, fatty acids are aliphatic carboxylic acids comprising unbranched or branched, optionally hydroxylated, hydrocarbon radicals having from 4 to 40, preferably from 8 to 24 carbon atoms. The fatty acids used in the present disclosure present disclosure may be naturally occurring fatty acids or synthetically produced fatty acids. Furthermore, the fatty acids can be monounsaturated or polyunsaturated. The fatty acid may also contain a mixture of several fatty acids.

Particularly preferred fatty acids are including comprising selected from the group of behenic acid, fatty acids derived from borage seed oil (Borago officinalis L.), fatty acids derived from Vateria indica, 12-hydroxystearic acid, isostearic acid, fatty acids derived from meadowfoam seed oil, fatty acids of mahua butter, fatty acids derived from Shorea (sal) butter, fatty acids derived from coconut butter, fatty acids derived from illipe butter, stearic acid and mixtures thereof.

Reaction products of a hydroxy-terminated polyorganosiloxane with a fatty acid include, for example, the reaction product of dimethiconol with behenic acid (INCI: dimethiconol behenate), the reaction product of dimethiconol with fatty acids obtained from the seed oil of borage (Borago officinalis L.) (INCI: dimethiconol borageate), the reaction product of dimethiconol with fatty acids obtained from Vateria indica (INCI: dimethiconol dhupa butterate), the reaction product of dimethiconol with 12-hydroxystearic acid (INCI: dimethiconol hydroxystearate), the reaction product of dimethiconol with isostearic acid (INCI: dimethiconol isostearate), the reaction product of dimethiconol with fatty acids obtained from the seed oil of American meadowfoam (“meadowfoam seed oil”) (INCI: dimethiconol meadowfoamate), the reaction product of dimethiconol with fatty acids derived from mohwa butter (INCI: dimethiconol mohwa butterate), the reaction product of dimethiconol with fatty acids derived from Shorea (sal) butter (INCI: dimethiconol sal butterate), the reaction product of dimethiconol with fatty acids derived from coconut butter (INCI: dimethiconol kokum butterate), the reaction product of dimethiconol with fatty acids obtained from illipe butter (INCI: dimethiconol illipe butterate) and/or the reaction product of dimethiconol with stearic acid (INCI: dimethiconol stearate).

It is much preferred if the fatty acid is including comprising selected from the group of fatty acids derived from meadowfoam seed oil, behenic acid, stearic acid and mixtures thereof. In particular, it is preferred that the fatty acid comprises a mixture of fatty acids derived from meadowfoam seed oil. It is highly preferred that the fatty acid is a mixture of fatty acids derived from meadowfoam seed oil.

It is preferred that the amino acid is including comprising selected from the group of glycine, alanine, valine, isoleucine, leucine, phenylalanine, tyrosine, lysine, arginine, histidine, aspartic acid, glutamic acid, threonine, serine, glutamine, asparagine, methionine, cysteine, proline, tryptophan, cystine, ornithine, citrulline, and mixtures thereof. It is much preferred if the amino acid is including comprising selected from the group of arginine, methionine, cysteine and mixtures thereof.

Reaction products of a hydroxy-terminated polyorganosiloxane with an amino acid include, for example, the reaction product of dimethiconol with arginine (INCI: dimethiconol arginine), the reaction product of dimethiconol with methionine (INCI: dimethiconol methionine), the reaction product of dimethiconol with cysteine (INCI: dimethiconol cysteine), or mixtures thereof.

Suitable α-hydroxy acids include, in particular, lactic acid.

For example, a reaction product of a hydroxy-terminated polyorganosiloxane with an α-hydroxy acid comprises the reaction product of dimethiconol with lactic acid (INCI: dimethiconol lactate).

The alcohol is preferably including comprising selected from the group of fatty alcohols and polyols.

In the context of the present disclosure present disclosure, a fatty alcohol means aliphatic, monovalent, primary alcohols, which comprises unbranched or branched hydrocarbon radicals having 4 to 40, preferably 8 to 24 carbon atoms. The fatty alcohols used in the present disclosure may also be mono- or polyunsaturated.

A suitable polyol comprises in particular dexpanthenol (INCI: panthenol).

For example, a reaction product of a hydroxy-terminated polyorganosiloxane with a polyol comprises the reaction product of dimethiconol with dexpanthenol (INCI: dimethiconol panthenol).

Natural waxes in particular can be used as waxes. Suitable vegetable waxes preferably include candelilla wax (Euphorbia cerifera (Candelilla) wax) and/or carnauba wax (INCI: Copernicia cerifera (Carnauba) wax). Animal waxes such as beeswax (INCI: Cera Alba) may also be used.

Reaction products of a hydroxy-terminated polyorganosiloxane with a wax include, for example, the reaction product of dimethiconol with candelilla wax (INCI: dimethiconol candelillate), the reaction product of dimethiconol with carnauba wax (INCI: dimethiconol carnaubate) and/or the reaction product of dimethiconol with beeswax (INCI: dimethiconol beeswax).

Of all the above reaction products of a hydroxy-terminated polyorganosiloxane with an acid and/or an alcohol and/or a wax, the most preferred are the reaction product of dimethiconol with stearic acid (INCI: dimethiconol stearate) and/or the reaction product of dimethiconol with fatty acids obtained from meadowfoam seed oil (INCI: dimethiconol meadowfoamate). Use of the reaction product of dimethiconol with fatty acids obtained from meadowfoam seed oil (INCI: dimethiconol meadowfoamate) is most preferred.

The reaction product of dimethiconol with fatty acids obtained from the seed oil of American meadowfoam seed oil (INCI: dimethiconol meadowfoamate) is available, for example, under the name “Fancorsil LIM-2” from Elementis.

The reaction product of dimethiconol with stearic acid (INCI: dimethiconol stearate) is available for example under the name “SilSense® IWS silicone” from Lubrizol.

The cosmetic product comprises the reaction product of a hydroxy-terminated polyorganosiloxane with an acid and/or an alcohol and/or a wax, in an amount of from 0.1 to 10% by weight, preferably from 0.2 to 8% by weight, more preferable from 0.3 to 6% by weight, greatly preferable from 0.4 to 4% by weight and particularly preferable from 0.5 to 2% by weight, in each case based on the weight of the cosmetic product.

The ratio of hydroxy-terminated polyorganosiloxane to the reaction product of a hydroxy-terminated polyorganosiloxane with an acid and/or an alcohol and/or a wax is preferably in the range 5:1 to 1:5, and more preferably in the range 3:1 to 1:3.

Surprisingly, the combination of a hydroxy-terminated polyorganosiloxane and a reaction product of a hydroxy-terminated polyorganosiloxane with an acid and/or an alcohol and/or a wax has been shown to result in intense conditioning of keratinous fibers, particularly human hair. In particular, the hydrophobicity of severely damaged hair is improved, bringing it back closer to its original natural state.

The cosmetic product further comprises an aqueous carrier or an aqueous-alcoholic carrier. An aqueous carrier comprises at least 50% by weight, more preferable at least 60% by weight. An aqueous alcoholic carrier is understood to be aqueous solutions comprising 3 to 70% by weight of a C2-C6 alcohol, in particular ethanol or propanol, isopropanol, butanol, isobutanol, tert-butanol, n-pentanol, iso-pentanols, n-hexanol, iso-hexanols, glycol, glycerol, 1,2-pentanediol, 1,5-pentanediol, 1,2-hexanediol or 1,6-hexanediol. The agents may additionally contain other organic solvents, such as methoxybutanol, benzyl alcohol, ethyl diglycol or 1,2-propylene glycol. Preferred are all water-soluble organic solvents. Preferably, the cosmetic product comprises an aqueous carrier comprising 100% by weight of water. The amount of the aqueous carrier is preferably at least 80% by weight, based on the total weight of the cosmetic product.

Preferred cosmetic products are identified as follows:
0.1 to 8% by weight of a hydroxy-terminated polyorganosiloxane,
0.2 to 8% by weight of a reaction product of a hydroxy-terminated polyorganosiloxane with an acid and/or an alcohol and/or a wax, and
an aqueous carrier.

More preferred cosmetic products are further exemplified as follows:
0.2 to 2% by weight of a hydroxy-terminated polyorganosiloxane,
0.4 to 4% by weight of a reaction product of a hydroxy-terminated polyorganosiloxane with an acid and/or an alcohol and/or a wax comprising the reaction product of a hydroxy-terminated polyorganosiloxane with an acid, and
an aqueous carrier.

Even more preferred cosmetic products are exemplified as follows:
0.2 to 2% by weight of a hydroxy-terminated polyorganosiloxane,
0.4 to 4% by weight of a reaction product of a hydroxy-terminated polyorganosiloxane with an acid and/or an alcohol and/or a wax comprising the reaction product of a hydroxy-terminated polyorganosiloxane with a fatty acid, and
an aqueous carrier.

Still further preferred cosmetic products are exemplified as follows:
0.2 to 2% by weight of a hydroxy-terminated polyorganosiloxane comprising at least one compound known under the INCI name amodimethicone/morpholinomethyl silsesquioxane copolymer,
0.4 to 4% by weight of a reaction product of a hydroxy-terminated polyorganosiloxane with an acid and/or an alcohol and/or a wax comprising the reaction product of a hydroxy-terminated polyorganosiloxane with a fatty acid, and
an aqueous carrier.

Even more preferred cosmetic products are exemplified as follows:
0.2 to 2% by weight of a hydroxy-terminated polyorganosiloxane comprising at least one compound known under the INCI name amodimethicone/morpholinomethyl silsesquioxane copolymer,
0.4 to 4% by weight of a reaction product of a hydroxy-terminated polyorganosiloxane with an acid and/or an alcohol and/or a wax comprising the reaction product of a hydroxy-terminated polyorganosiloxane with fatty acids derived from meadowfoam seed oil, and
an aqueous carrier.

Highly preferred cosmetic products are exemplified as follows:
0.2 to 2% by weight of a hydroxy-terminated polyorganosiloxane comprising at least one compound known under the INCI name amodimethicone/morpholinomethyl silsesquioxane copolymer,
0.4 to 4% by weight of a reaction product of a hydroxy-terminated polyorganosiloxane with an acid and/or an alcohol and/or a wax comprising the reaction product of dimethiconol with fatty acids obtained from meadowfoam seed oil (INCI: dimethiconol meadowfoamate) and
an aqueous carrier.

In the following, further ingredients of the cosmetic products are described which may be included in the products in addition to the mandatory ingredients described above.

Other suitable ingredients include nonionic surfactants, nonionic polymers, protein hydrolysates, oligopetides, vitamins, provitamins, vitamin precursors, betaines, bioquinones, purine (derivatives), taurine (derivatives), plant extracts, other silicones, ester oils, UV light filters, structuring agents, thickening agents, electrolytes, pH adjusting agents, swelling agents, colorants, complexing agents, opacifiers, pearlescent agents, pigments, stabilizing agents, antioxidants, perfume oils and/or preservatives.

It is preferred that the cosmetic product has a pH in the range of 2.5 to 6.5, preferably 2.5 to 5.5, particularly preferable 2.5 to 4.5 and especially preferable 2.5 to 3.5. Mineral acids or organic acids may be used to adjust the pH, organic acids being preferred. The organic acid is preferably including comprising selected from the group of maleic acid, lactic acid, acetic acid, propanoic acid, amino acids and mixtures thereof. Suitable amino acids include glycine, glutamic acid, arginine and/or aspartic acid. Lactic acid is particularly preferred for adjusting the pH value. The cosmetic products preferably contain the organic acid in an amount of from 0.1 to 5% by weight, and particularly preferably from 0.25 to 3% by weight, based in each case on the weight of the cosmetic product.

It is particularly preferred that the cosmetic product includes a fatty alcohol. The fatty alcohol acts as an emulsifier, consistency enhancer and/or thickener.

The fatty alcohol may be selected, for example, from C9-C11 fatty alcohols, C12-C13 fatty alcohols, C12-C15 fatty alcohols, C12-C16 fatty alcohols, C14-C15 fatty alcohols, arachidyl alcohol, behenyl alcohol, caprylic alcohol, cetearyl alcohol, cetyl alcohol, coconut alcohol, decyl alcohol, (hydrogenated) tallow alcohol, lauryl alcohol, myristyl alcohol, oleyl alcohol, palm alcohol, palm kernel alcohol, stearyl alcohol and/or tridecyl alcohol.

In a particularly preferred embodiment of the cosmetic products, the fatty alcohol includes cetearyl alcohol.

The amount of the fatty alcohol is preferably 0.5 to 10% by weight, more preferable 1 to 9% by weight and in particular preferable 2 to 8% by weight, in each case based on the total amount of cosmetic product.

It may be further preferred that the cosmetic product comprises an alkoxylated fatty alcohol. The alkoxylated fatty alcohol acts as an emulsifier.

The fatty alcohol may be selected, for example, from C9-C11 fatty alcohols, C12-C13 fatty alcohols, C12-C15 fatty alcohols, C12-C16 fatty alcohols, C14-C15 fatty alcohols, arachidyl alcohol, behenyl alcohol, caprylic alcohol, cetearyl alcohol, cetyl alcohol, coconut alcohol, decyl alcohol, (hydrogenated) tallow alcohol, lauryl alcohol, myristyl alcohol, oleyl alcohol, palm alcohol, palm kernel alcohol, stearyl alcohol and tridecyl alcohol.

In particular, the alkoxy group may include ethoxy and/or propoxy and/or butoxy groups. It would be particularly preferred if the alkoxylated fatty alcohol is an ethoxylated fatty alcohol.

At least one alkoxy group of the “alkoxylated fatty alcohol” may, for example, be derived from an alkoxylation reaction with alkylene oxide, in particular ethylene oxide and/or propylene oxide.

It is preferred that the alkoxylated fatty alcohol is including comprising selected from the group of ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, ceteareth-40, ceteareth-50, ceteareth-55, ceteareth-60, ceteareth-80, ceteareth-100, laureth-1, laureth-2, laureth-3, laureth-4, laureth-5, laureth-6, laureth-7, laureth-8, laureth-9, laureth-10, laureth-11, laureth-12, laureth-13, laureth-14, laureth-15, laureth-16, laureth-20, laureth-23, laureth-25, laureth-30, laureth-40, deceth-3, deceth-5, oleth-5, oleth-30, steareth-2, steareth-4, steareth-6, steareth-7, steareth-10, steareth-11, steareth-13, steareth-14, steareth-15, steareth-20, steareth-21, steareth-25, steareth-27, steareth-30, steareth-40, steareth-50, steareth-100 and mixtures thereof.

The designation ceteareth-2, for example, stands for a C16-C18 fatty alcohol with an average of 2 ethylene oxide units per molecule.

In a very particularly preferred embodiment of the cosmetic product, the alkoxylated fatty alcohol comprises Ceteareth-20.

The amount of the alkoxylated fatty alcohol is preferably 0.1 to 5% by weight and more preferable 0.25 to 3% by weight, in each case based on the total amount of cosmetic product.

It is particularly preferred that the agents are free of cationic compounds, as these can have a destabilizing effect. “Free of” means that the cosmetic product comprises a maximum of 0.2% by weight and preferably 0% by weight, in each case based on the total weight of the agent, of cationic compounds. Cationic compounds include, in particular, cationic surfactants such as quaternary ammonium compounds, esterquats and/or amidoamines, and cationic polymers. Cationic polymers are polymers which have at least one cationic group. This cationic group can be either permanently cationic or temporarily cationic. The term “permanently cationic” refers to groups that have a cationic group regardless of the pH of the cosmetic product. This includes in particular groups with quaternary nitrogen atoms, such as quaternary ammonium groups. On the other hand, “temporarily cationic” groups are those which have a cationic group only at certain pH values, especially pH values in the acidic range. Examples of temporary cationic groups are amine groups.

The cosmetic agents described are used to improve the condition of keratinous fibers treated with them. This includes, for example, improving the condition of the keratinous fibers by hardening, strengthening, sealing (especially after dyeing or toning), restructuring, repairing, stabilizing, smoothing the surface, protecting against environmental influences, protecting against heat (when blow-drying and/or straightening with a hot iron) and increasing the resilience and elasticity of the keratinous fibers. In particular, improving the condition of keratinous fibers includes hydrophobizing severely damaged keratinous fibers, especially human hair.

Another object of the present disclosure is a method for treating keratinous fibers, in which a cosmetic product present disclosure as contemplated herein is applied to keratinous fibers, in particular human hair, and is rinsed off the keratinous fibers after a contact time.

The exposure time is preferably from a few seconds to 45 minutes, more preferable from 0.5 to 15 minutes and most preferable from 1 to 5 minutes.

The following examples are intended to explain the subject matter of the present disclosure present disclosure without, however, limiting it.

### EXAMPLES

All quantities are parts by weight unless otherwise stated. The following formulations were provided using known manufacturing procedures.

### Cosmetic Products E1 to E4:

| | E1 | E2 | E3 | E4 |
|---|---|---|---|---|
| Belsil ® ADM 8301 E* | 5 | 5 | 5 | 5 |
| Fancorsil LIM-2** | 0.5 | 0.5 | — | — |
| SilSense ® IWS Silicone*** | — | — | 0.5 | 0.5 |
| Lactic acid | 0.6 | 0.6 | 0.6 | 0.6 |
| Cetearyl alcohol (INCI) | — | 5 | — | 5 |
| Ceteareth-20 (INCI) | — | 1 | — | 1 |
| Water | ad 100 | ad 100 | ad 100 | ad 100 |

*Active silicone content: 27%-31% by weight
**Active silicone content: 100% by weight
***Active silicone content: 100% by weight

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product for the treatment of keratinous fibers, containing (relative to the weight of the entire agent)
a) 0.1 to 10% by weight of a hydroxy-terminated polyorganosiloxane,
b) 0.1 to 10% by weight of a reaction product of a hydroxy-terminated polyorganosiloxane with an acid and/or a alcohol and/or a wax, and
c) an aqueous or aqueous-alcoholic carrier.

2. The cosmetic product according to claim 1, characterized in that the agent contains, as hydroxy-terminated polyorganosiloxane, a polyorganosiloxane of the formula (I) 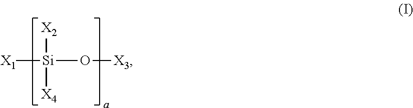
a includes,
wherein
X1 and X2 are independently OH, OR1, R2, O-PDMS or O-fSiloxane,
X3 is hydrogen or a monovalent hydrocarbon radical having 1 to 8 carbon atoms per radical, PDMS or fsiloxane,
X4 is a remainder of the formula 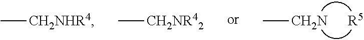
and
a is a number from 1 to 100,
where
R1 is an alkyl radical having 1 to 8 carbon atoms,
R2 denotes a monovalent, saturated or unsaturated hydrocarbon radical which is optionally substituted by the elements N, P, S, O, Si and halogen and has 1 to 200 carbon atoms per radical, 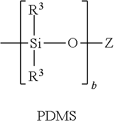
means, 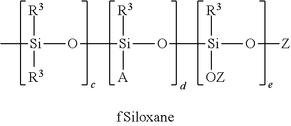
means,
R3 independently of one another is in each case a monovalent saturated or unsaturated hydrocarbon radical having 1 to 200 carbon atoms per radical and optionally substituted by the elements N, P, S, O, Si and halogen,
A is a radical of the formula R6-[NR7-R8-]fNR72,
where
R6 represents a divalent linear or branched hydrocarbon radical containing 3 to 18 carbon atoms,
R7 represents a hydrogen atom, an alkyl group having 1 to 8 carbon atoms or an acyl group,
R8 is a divalent hydrocarbon radical having 1 to 6 carbon atoms,
b is a number from 1 to 2000,
c is 0 or a number from 1 to 2000,
d is a number from 1 to 1000,
e is 0 or a number from 1 to 5,
f is 0, 1, 2, 3 or 4,
Z is hydrogen, an alkyl radical having 1 to 8 carbon atoms or 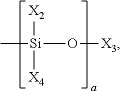
R4 represents a monovalent hydrocarbon radical optionally containing N and/or O atoms and having 1 to 18 carbon atoms, and
R5 represents a divalent hydrocarbon radical optionally containing N and/or O atoms and having 3 to 12 carbon atoms,
with the proviso that the polyorganosiloxane of formula (I) has at least one terminal OH group.

3. The cosmetic product according to claim 2, characterized in that fSiloxane is an amine-functionalized siloxane, preferably a copolymer of 3-(2-aminoethylamino)propylmethylsiloxy and dimethylsiloxy units.

4. The cosmetic product according to claim 2, characterized in that the polyorganosiloxane of the formula (I) contains, as radical X4, a morpholinomethyl radical.

5. The cosmetic product according to claim 1, characterized in that the cosmetic product contains, as hydroxy-terminated polyorganosiloxane, at least one compound known under the INCI designation amodimethicone/morpholinomethyl silsesquioxane copolymer.

6. The cosmetic product according to claim 1, characterized in that the cosmetic product contains a reaction product of a hydroxy-terminated polyorganosiloxane with an acid selected from the group consisting of reaction products of a hydroxy-terminated polyorganosiloxane with a fatty acid, reaction products of a hydroxy-terminated polyorganosiloxane with an amino acid, reaction products of a hydroxy-terminated polyorganosiloxane with an α-hydroxy acid, and mixtures thereof.

7. The cosmetic product according to claim 6, characterized in that the agent contains a reaction product of a hydroxy-terminated polyorganosiloxane with a fatty acid selected from the group consisting of reaction products of dimethiconol with fatty acids derived from meadowfoam seed oil (INCI: dimethiconol meadowfoamate), reaction products of dimethiconol with stearic acid (INCI: dimethiconol stearate), and mixtures thereof.

8. The cosmetic product according to claim 1, characterized in that the cosmetic product further contains branched ethoxylated tridecanol (INCI name: trideceth-5) or α-iso-tridecyl-ω-hydroxypolyglycol ether (INCI name: trideceth-10) or mixtures thereof.

9. The cosmetic product according to claim 1, characterized in that the agent is free of cationic compounds, in particular cationic surfactants.

10. The cosmetic product according to claim 1, characterized in that the cosmetic product has a pH in the range from 2.5 to 6.5, preferably 2.5 to 5.5, particularly preferable 2.5 to 4.5, and especially preferable 2.5 to 3.5.

11. The cosmetic product according to claim 1, characterized in that the cosmetic product further contains a fatty alcohol.

12. The cosmetic product of claim 1, characterized in that the cosmetic product further contains an alkoxylated fatty alcohol.

13. (canceled)

14. (canceled)
